(19) United States
(12) Patent Application Publication
(10) Pub. No.: US 2010/0055776 A1
(43) Pub. Date: Mar. 4, 2010

(54) BIOREACTOR, ARRANGEMENT OF BIOREACTORS, PROCESS FOR PRODUCTION THEREOF AND USE THEREOF

(75) Inventors: Karl-Friedrich Weibezahn, Stutensee (DE); Eric Gottwald, Karlsruhe (DE); Roman Truckenmueller, Flein (DE); Stefan Giselbrecht, Karlsruhe (DE)

Correspondence Address: VENABLE LLP, P.O. BOX 34385, WASHINGTON, DC, 20043-9998, US

(73) Assignee: FORSCHUNGSZENTRUM KARLSRUHE GmbH., KARLSRUHE (DE)

(21) Appl. No.: 12/527,518

(22) PCT Filed: Feb. 12, 2008
(86) PCT No.: PCT/EP08/01048

§ 371 Date: Aug. 17, 2009

(30) Foreign Application Priority Data
Feb. 16, 2007 (DE) 10 2007 007 718.3

(51) Int. Cl. C12M 1/12; C12M 1/00; B29C 45/00

(52) U.S. Cl. 435/297.1; 435/289.1; 264/328.1

(57) ABSTRACT

The invention relates to a bioreactor including a housing having a lower housing half and an upper housing half that are respectively connected to one another on one side by a film joint. At least one of the upper or lower housing halves includes a device to exchange fluids. The bioreactor further includes at least one support element to support at least one cell culture substrate. The invention furthermore relates to an arrangement of such bioreactors, processes for production thereof and use thereof for culturing cells or as an implant.

### CROSS-REFERENCE TO RELATED APPLICATION

This application is a National Stage application of International Application No. PCT/EP2008/001048, filed on Feb. 12, 2008 and designating the United States.

### BACKGROUND OF THE INVENTION

The invention relates to a bioreactor for the cultivation of cells, in particular for the long-term cultivation of cells, an arrangement consisting of such bioreactors, a process for the production thereof and the use thereof.

Currently used bioreactors are composed of a plurality of parts, wherein the cell culture substrates are not connected to the bioreactor but are inserted separately. In addition, these parts are normally produced from different materials which for the most part require different production methods.

The German patent document DE 41 32 379 C2 discloses a substrate for cell cultures that consists of a plate-shaped body provided with a plurality of micro-containers, separated from each other by webs, for accommodating the cells or the cell agglomerates.

The German patent document DE 10 2004 035 267 B3 discloses a formed body consisting of a film with at least one cavity and a plurality of pores incorporated therein, as well as the use of this body for the cultivation of cells and as a bioreactor.

The German patent document 20 2006 012 978 U1 describes a bioreactor comprising a base plate with a recess for accommodating a support structure with pores for cell cultures, an upper and a lower transparent cover film, as well as conduits for supplying the cell cultures located on the support structure.

Thermo-formed folding boxes are used in many different ways, in particular in the food sector. For this, two half shells or so-called trays, meaning a lower and an upper shell half or a lower shell half and an associated lid, are connected to each other via a film joint, wherein these parts are typically shaped from the same film. Similar to a crease, the goal of a suitable shaping of a film joint is to have a defined bend or fold along a straight line in the original film plane. The re-closable connection of the folded up parts is realized, for example, by pressing a circumferential ridge that is embodied in one shell half into a circumferential groove or depression embodied in the other shell half, or that a lid formed in the same direction but embodied flatter is clamped into a lower shell half. Slanted side walls in that case function as centering aids and to prevent an (automatic) reopening.

### SUMMARY

Starting with this premise, it is an object of the present invention to propose a bioreactor that can be produced easily and is easy to handle. Additionally, the bioreactor may also be suitable for a one-time use, so that it consequently does not have to be cleaned and sterilized following the use.

A bioreactor according to an embodiment in the form of a folding box includes a housing, having a lower housing half and an upper housing half that are joined on respectively one side by a film joint, as well as at least one support element for at least one cell culture substrate, wherein a device for exchanging fluids is provided on at least one of the two housing halves.

According to an embodiment, the at least one support element is inserted between the lower housing half and the upper housing half to come to rest between the two housing halves when these are in the folded up state.

According to an alternative embodiment, the at least one support element is inserted into the lower housing half or into the upper housing half to form a component of the respective housing half or to fixedly connect thereto.

The bioreactor according to another embodiment includes two support elements, of which one support element is a component of one of the two housing halves while the other support element is inserted between the two housing halves. This type of embodiment permits, for example, the cultivation of co-cultures.

The lower housing half and the upper housing half may be fluid-tight relative to each other, meaning they are impermeable to liquids or gas. According to an embodiment, the at least one support element is arranged fluid-tight relative to one of the two housing halves or between both housing halves. The at least one support element is provided for this with a circumferential seal that is effective toward the respective housing half and, if applicable, toward additional support elements. Alternatively, the function of joining the parts and sealing the parts against each other can also be realized with separate function elements.

According to another embodiment, the two housing halves and the at least one support element are arranged in a row or diagonally before being folded up. The number of sealing interfaces increases along with an increasing number of support elements, which in practice means they are restricted to just a few.

The bioreactor according to one embodiment is configured in the manner of a flow cell. At least one of the two housing halves may be provided with one, two or a plurality of fluid connections for supplying fluid with therein dissolved nourishment and gases to the cells anchored in (adhering to) the at least one cell culture substrate, as well as to allow the removal of cell metabolism products via fluids in the form of a perfusion, a one-sided or two-sided super-fusion, or a mixed form thereof via a culture medium. As a result, physical and chemical gradients can be adjusted in the bioreactor via the cell culture substrate. The fluidic connections are located only on the top, respectively the bottom, or lateral, preferably not lateral, in the region of the fluid-tight connections.

At least one of the two housing halves for an alternative embodiment is provided with a permeable or a semi-permeable membrane or a porous region, wherein these respectively function to allow an exchange of fluids through perfusion or osmosis.

The core piece of the bioreactor according to an embodiment may be the at least one cell culture substrate, which is advantageously suitable for a three-dimensional cultivation of cells in the form of an arrangement of micro-containers, e.g. as disclosed in the German patent documents DE 41 32 379 C2 or DE 10 2004 03 5267 B3. Alternatively, the at least one cell culture substrate is suitable for a two-dimensional cultivation of cells in the form of a planar substrate.

The at least one cell culture substrate according to one embodiment is perforated to ensure an improved passive or active substance transport. Also conceivable in principle are micro-structured or nano-structured substrates. The cell culture substrate can implicitly (statistical) or explicitly (regular) have a micro structure or a nano structure.

There are many options for configuring the bioreactor with respect to the selection of the film material, the shape and dimensions of the bioreactor, the number of support elements respectively provided with one or a plurality of cell culture substrates, the arrangement of the upper housing half, the lower housing half and the support element or elements, the embodiment of the film joints, the means for exchanging fluids, as well as the size, number and shape of the cell culture substrates.

The heat-deformable, thermoplastic or thermo-elastic film material for the bioreactor may be selected in particular on the basis of the following aspects: bio-compatibility, physical-chemical resistance, transparence and low inherent fluorescence.

The bioreactor may be composed of polymethylmethacrylate (PMMA), polycarbonate (PC), polyethylene terephthalat (PET), polystyrene (PS), polyimide (PI), polyethylene (PE), polypropylene (PP), polyvinylfluoride (PVF), polyvinylidenfluoride (PVDF), polyvinylchloride (PVC), polyvinylidenchloride (PVDF), polyetherimide (PEI), polyetheretherketone (PEEK), polysulphone (PSU), polyurethane (PU), cycloolefincopolymer (COC) or cycloolefinpolymer (COP).

Alternatively, the bioreactor can also be composed of poly lactic acid (PLA), of poly (ε caprolactone), of a polyhydroxyalkanoate (PHA) or a different biodegradable polymer.

One embodiment furthermore includes an arrangement of connected units, including two or more and up to a plurality of bioreactors that are combined to form a single unit.

A bioreactor according to an embodiment, together with the cell culture substrate, may be injection-molded in the form of a folding box or may be hot formed, in particular thermoformed, as a single part from a single piece of film material, wherein the joints and the fluid-tight seals are formed on at the same time.

Depending on the type and thickness of the film material or the semi-finished product thereof, used for the bioreactor and t he shaping of the at least one support element, the at least one cell culture substrate according to one embodiment is formed into the same semi-finished product and during the same forming process as used for the lower housing half and the upper housing half, directly into the support element that is connected via a film joint to the housing halves.

According to one alternative embodiment, the at least one cell culture substrate is incorporated into a separate semi-finished product, which is then inserted between the upper housing half and the lower housing half. In the process, a fluid-tight connection is created between the at least one cell culture substrate and the two housing halves.

According to a different embodiment, the support element that is connected articulated via an additional film joint to the lower housing half, the upper housing half or, if applicable, to an additional support element, is embodied as a frame into which at least one cell culture substrate is inserted.

If the support element is connected articulated with the lower housing half or the upper housing half, it can be separated from the remaining bioreactor for the further processing by cutting or punching the film, respectively by tearing the film along a perforation line.

The hot-forming process used herein as a rule includes the so-called trimming, which generally is a finishing process, meaning the removal of the formed part from the remaining film material with the aid of punching, laser cutting, water-jet cutting or the like.

According to an embodiment, outside contours as well as inside contours can be created with this separating technique. With the aid of fluidic connections that are formed-on and stamped out or screwed to punched-out openings in the two housing halves, in a similar manner as described in the German patent document DE 20 2006 012 978 U1, a perfusion or superfusion of the cell culture substrate with culture medium can be achieved, or also a combination of the two modes.

The bioreactor and the arrangement may be configured with few parts, or with only a single part. As a result of this simple configuration, which is typical for thermo-formed and comparably thin-walled products, involving relatively small amounts of preferably a single type of material, the bioreactor and the arrangement according to this embodiment are suitable for use as disposable items from an ecological and economical point of view.

The bioreactor and the arrangement according to an embodiment permit a sterile cultivation of cells or they can serve as implant for human and animal bodies.

### BRIEF DESCRIPTION OF THE DRAWINGS

The present invention will be more readily understood from the following detailed description when read in conjunction with the accompanying drawings, in which:

FIGS. 1A-C are perspective representations of a bioreactor, according to an embodiment of the invention, with a single support element in the unfolded state in FIG. 1A, the partially folded up state in FIG. 1B and the completely folded up state in FIG. 1C;

FIG. 2 is a perspective representation of a bioreactor, according to an alternative embodiment of the invention, with three support elements in the folded out state.

### DETAILED DESCRIPTION

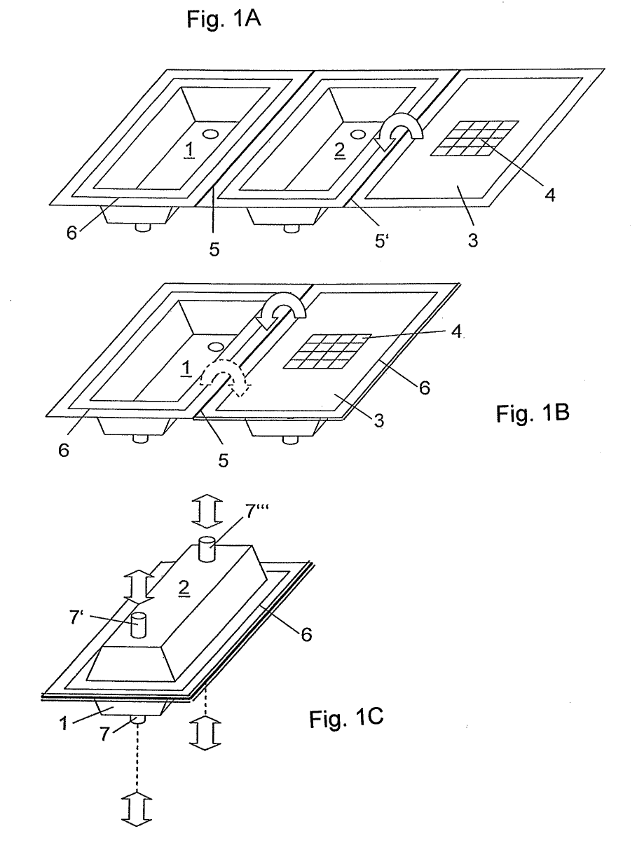

FIGS. 1A-C show perspective representations of a bioreactor according to the invention, including a lower housing half 1 and an upper housing half 2, respectively connected on one side with the aid of a film joint 5, and further including a support element 3 into which a cell culture substrate 4 is inserted. The bent arrows indicate the direction of folding and sealing. The bent arrow shown with dashed lines provides an alternative manner of folding, based on which the bioreactor is still standing on its head, which requires an additional turning around.

The support element which is connected via an additional film joint 5′ to the upper housing half 2 is provided with a circumferential sealing structure 6 that is adapted to the lower housing half 1 as well as the upper housing half 2.

The lower housing half 1 is provided with two fluid connections 7, 7″ (wherein the connection 7″ is not visible because of the perspective) and the upper housing half 2 is also provided with two fluid connections 7′, 7′″. The straight arrows indicate the flow-through direction in the superfusion mode, the perfusion mode, or the mixed mode.

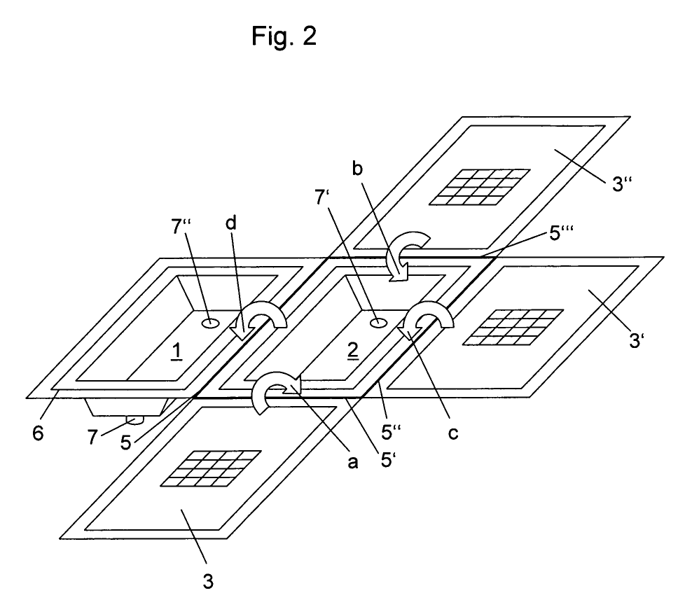

The perspective representation in FIG. 2 shows a bioreactor according to the invention, provided with three support elements 3, 3′, 3″ and four film joints 5, 5′, 5″, 5′″. The arrangement of the housing halves 1, 2 and the support elements 3, 3′, 3″ was selected with the idea of a simple tool design. The sequence for folding up the bioreactor is indicated with the bent arrows a, b, c, d.

It will be understood that the above description of the present invention is susceptible to various modifications, changes and adaptations, and that the same are intended to be comprehended within the meaning and range of equivalents of the appended claims.

1. A bioreactor, comprising:
a housing having a lower housing half and an upper housing half that are respectively connected to one another on one side by a film joint, wherein at least one of the upper or lower housing halves includes a device to exchange fluids; and
at least one support element to support at least one cell culture substrate in the housing.

2. The bioreactor according to claim 1, wherein the at least one support element is inserted into one of the lower housing half or the upper housing half.

3. The bioreactor according to claim 1, wherein the at least one support element is inserted between the lower housing half and the upper housing half.

4. The bioreactor according to claim 1, wherein the lower housing half and the upper housing half are arranged to be fluid-tight relative to each other.

5. The bioreactor according to claim 4, wherein the at least one support element is arranged to be fluid-tight, relative to at least one of the upper or lower housing halves.

6. The bioreactor according to claim 3, wherein the at least one support element is connected on one side by a second film joint to one of the lower housing half or the upper housing half, and wherein the at least one support element is foldable in via the second film joint to be positioned between the lower housing half and the upper housing half.

7. The bioreactor according to claim 6, further comprising at least a second support element connected via a third film joint to one of the lower housing half, the upper housing half, or the at least one support element.

8. The bioreactor according to claim 1, wherein at least one of the upper or lower housing halves includes at least one connection for the exchange of fluids.

9. The bioreactor according to claim 1, wherein at least one of the upper or lower housing halves includes one of a permeable membrane, a semi-permeable membrane, or a porous region for the exchange of fluids.

10. The bioreactor according to claim 1, wherein the at least one cell culture substrate is formed into the at least one support element.

11. The bioreactor according to claim 1, wherein the at least one support element comprises a frame into which the at least one cell culture substrate is inserted.

12. The bioreactor according to claim 1, wherein the at least one cell culture substrate is perforated.

13. The bioreactor according to claim 1, wherein the housing comprises one of a hot-formed, thermoplastic or thermo-elastic film material.

14. The bioreactor according to claim 13, wherein the housing comprises one of polymethylmethacrylate, polycarbonate, polyethylene terephthalate, polystyrene, polyimide, polyethylene, polypropylene, polyvinylfluoride, polyvinylidenfluoride, polyvinylchloride, polyvinylidenchloride, polyetherimide, polyetheretherketone, polysulphone, polyurethane, cycloolefin copolymer or cycloolefin polymer.

15. The bioreactor according to claim 13, wherein the housing comprises one of poly lactic acid, poly(ε-caprolactone), a polyhydroxyalkanoate or a different biodegradable polymer.

16. An arrangement comprising at least two connected bioreactors in accordance with claim 1.

17. A method for producing a bioreactor, comprising producing the bioreactor claim 1, by one of injection-molding or thermoforming from a piece of film material.

18. The method according to claim 17, further comprising forming at least one cell culture substrate in a same semi-finished product and during a same forming process used for positioning at least one of the upper or lower housing halves into the at least one support element, wherein the at least one support element is connected to the upper or lower housing half via a film joint.

19. The method according to claim 17, further comprising forming the at least one cell culture substrate in a separate semi-finished product, and inserting the separate semi-finished product between the lower housing half and the upper housing half.

20. A method for cultivating cells or for using cells as an implant, comprising utilizing the bioreactor of claim 1.
